(19) United States
(12) Patent Application Publication
(10) Pub. No.: US 2011/0306997 A9
(43) Pub. Date: Dec. 15, 2011

(54) DEVICES FOR CREATING PASSAGES AND SENSING FOR BLOOD VESSELS

(76) Inventors: Edmund J. ROSCHAK, Mission Viejo, CA (US); Thomas M. Keast, Sunnyvale, CA (US); Patrick John Massetti, Mountain View, CA (US)

(21) Appl. No.: 11/562,947

(22) Filed: Nov. 22, 2006

Related U.S. Application Data
(63) Continuation-in-part of application No. 10/080,344, filed on Feb. 21, 2002, now Pat. No. 7,422,563.
(60) Provisional application No. 60/739,153, filed on Nov. 22, 2005.

(51) Int. Cl. A61B 17/34

(52) U.S. Cl. 606/185; 606/191; 623/23.72

(57) ABSTRACT

Devices and methods are disclosed for creating passages in tissue and detecting blood vessels in and around the passages. The devices may be used to create channels for altering gaseous flow within a lung to improve the expiration cycle of an individual, particularly individuals having Chronic Obstructive Pulmonary Disease (COPD). In addition, the devices may be used to sample tissue during biopsy or other medical procedures where perforating a blood vessel could result in injury to a patient.

### FIELD OF THE INVENTION

The invention is directed to devices for creating passages in tissue and detecting blood vessels in and around the passages. The device may be used to create channels for altering gaseous flow within a lung to improve the expiration cycle of an individual, particularly individuals having Chronic Obstructive Pulmonary Disease (COPD). In addition, the device may be used to sample tissue during biopsy or other medical procedures where perforating a blood vessel could result in injury to a patient.

### BACKGROUND OF THE INVENTION

The American Lung Association (ALA) estimates that nearly 16 million Americans suffer from chronic obstructive pulmonary disease (COPD) which includes diseases such as chronic bronchitis, emphysema, and some types of asthma. The ALA estimated that COPD was the fourth-ranking cause of death in the U.S. The ALA estimates that about 14 million and 2 million Americans suffer from emphysema and chronic bronchitis respectively.

Those inflicted with COPD face disabilities due to the limited pulmonary functions. Usually, individuals afflicted by COPD also face loss in muscle strength and an inability to perform common daily activities. Often, those patients desiring treatment for COPD seek a physician at a point where the disease is advanced. Since the damage to the lungs is irreversible, there is little hope of recovery. Most times, the physician cannot reverse the effects of the disease but can only offer treatment and advice to halt the progression of the disease.

Lung volume reduction surgery is a procedure which removes portions of the lung that are over-inflated. The portion of the lung that remains has relatively better elastic recoil, providing reduced airway obstruction. The reduced lung volume also improves the efficiency of the respiratory muscles. However, lung reduction surgery is an extremely traumatic procedure which involves opening the chest and thoracic cavity to remove a portion of the lung. As such, the procedure involves an extended recovery period. Hence, the long term benefits of this surgery are still being evaluated. In any case, it is thought that lung reduction surgery is sought in those cases of emphysema where only a portion of the lung is emphysematous as opposed to the case where the entire lung is emphysematous. In cases where the lung is only partially emphysematous, removal of a portion of emphysematous lung which was compressing healthier portions of the lung allows the healthier portions to expand, increasing the overall efficiency of the lung. If the entire lung is emphysematous, however, removal of a portion of the lung removes gas exchanging alveolar surfaces, reducing the overall efficiency of the lung. Lung volume reduction surgery is thus not a practical solution for treatment of emphysema where the entire lung is diseased.

Both bronchodilator drugs and lung reduction surgery fail to capitalize on the increased collateral ventilation taking place in the diseased lung. There remains a need for a medical procedure that can alleviate some of the problems caused by COPD. There is also a need for a medical procedure that alleviates some of the problems caused by COPD irrespective of whether a portion of the lung, or the entire lung is emphysematous. The production and maintenance of collateral openings through an airway wall allows air to pass directly out of the lung tissue responsible for gas exchange. These collateral openings serve to decompress hyper inflated lungs and/or facilitate an exchange of oxygen into the blood.

It was found that creation of collateral channels in COPD patients allowed expired air to pass out of the lungs and decompressed hyper-inflated lungs. Such methods and devices for creating and maintaining collateral channels are discussed in U.S. Pat. No. 6,692,494; U.S. patent application Ser. Nos. 09/947,144, 09/946,706, and 09/947,126 all filed on Sep. 4, 2001; U.S. patent application Ser. No. 10/235,240 filed on Sep. 4, 2002; each of which is incorporated by reference herein in its entirety.

The creation of these channels also seems to overcome the shortcomings associated with bronchodilator drugs and lung volume reduction surgery. Placement of an implant within the channel further increased the duration of the treatment.

However, because creation of the opening/channel is typically performed within the airway under bronchoscopic observation, care must be taken so as not to rupture a pulmonary vessel that lies beneath or outside of the airway wall. The need to avoid rupturing vessels that may be hidden by the airway walls is also evident when a surgeon attempts obtains a biopsy sample from within the bronchial tree. In addition, because the pattern of the pulmonary vessels varies between patients, care must also be taken when working within the channel or biopsy site. For instance, although a channel may be created without puncturing a blood vessel, the subsequent dialation, insertion of an implant, and/or removal of biopsy material may perforate vessels that were otherwise undetected during the creation of the channel.

In view of the above, a need remains to increase the safety when creating openings in tissue so as not to rupture a blood vessel.

### SUMMARY OF THE INVENTION

The invention relates to creation of passages and/or removal of tissue while allowing sensing of blood vessels that may be in or around the area of the passage. Although specific reference is made to use of the subject invention within the lungs, it is noted that the invention may also be used within various other parts of the body that have a need for such safety measures.

The device allows for creating passages in tissue and sensing blood vessels in or around the passages. The device includes an elongate member having a near end and a far end, the far end including a tissue piercing member having a sharp tip able to penetrate soft tissue; and an ultrasound transducer assembly coupled to the tissue piercing member such that the ultrasound transducer assembly may axially move relative to the tissue piercing member. It is noted that either the elongate member or the tissue piercing member can be configured to be moveable within the other piece.

The device described herein may include a blunt tip that is coupled to the ultrasound transducer assembly such that when the blunt tip is moved at least adjacent to or distal to the sharp tip, the tissue piercing member is unable to penetrate soft tissue.

In an additional variation, the device described above may also include an expandable member, such as a balloon or other mechanical means. When used in the lungs, the expandable member may comprise a balloon. The balloon may be constructed out of a distensible (or elastic) material. Alternatively, the balloon may be constructed form a non-distensible material. Such a material may be desirable when attempting to dilate strong or tough tissue. The balloon may also include an additional transducer assembly that permits scanning of the tissue before, after, or during dilation of an opening in tissue.

The inventive device is configured to communicate with an analyzing device or control unit adapted to recognize the reflected signal or measure the Doppler shift between the signals. As mentioned herein, the source signal may be reflected by changes in density between tissue. In such a case, the reflected signal will have the same frequency as the transmitted signal. When the source signal is reflected from blood moving within a vessel, the reflected signal has a different frequency than that of the source signal. This Doppler Effect permits determination of the presence or absence of a blood vessel within tissue. The device may include a user interface which allows the user to determine the presence or absence of a blood vessel at the target site. Typically, the user interface provides an audible confirmation signal. However, the confirmation signal may be manifested in a variety of ways (e.g., light, graphically via a monitor/computer, etc.)

Although depicted as being external to the device, it is contemplated that the analyzing device may alternatively be incorporated into the device. The transducer assembly of the invention is intended to include any transducer assembly that allows for the observation of Doppler Effect, e.g., ultrasound, light, sound etc.

The invention also includes a method of treating lung tissue, method comprising selecting an area in lung tissue, examining the area of the lung tissue for the presence or absence of blood vessels, creating an opening in lung tissue; and examining the opening in the lung tissue for the presence or absence of blood vessels.

Examining the opening in the lung tissue may comprises inserting an ultrasound device into the opening in lung tissue to further identify the presence or absence of blood vessels beneath the surface of the lung tissue. Examination of the area of lung tissue for the presence of blood vessels may include examining the area at a surface of the lung tissue with the ultrasound device.

The opening may be expanded with a member such as a balloon. As noted, a non-distensible balloon may allow for greater pressurization during the expansion of tissue.

### BRIEF DESCRIPTION OF THE DRAWINGS

FIGS. 1A-1C illustrate various states of the natural airways and the blood-gas interface.

FIG. 1D illustrates a schematic of a lung demonstrating a principle of the effect of collateral channels placed therein.

FIGS. 2A-2D illustrates variations of the inventive device.

FIGS. 3A-3B illustrate additional variations of the inventive device having a blunt portion offset from a transducer assembly.

FIGS. 3C-3D illustrates additional variation of the device.

FIGS. 4A-4E additional configurations of the device.

FIGS. 5A-5C illustrate another variation of the device where the sharp tip and elongate member only partially cover the transducer assembly.

FIGS. 6A-6C illustrate the invention as having an expandable member.

FIGS. 7A-7C illustrate variations of components of the invention that assist in deploying the tissue piercing member.

FIGS. 8A-8B illustrate a non-exhaustive sample of variations of the transducer assembly.

FIGS. 9A-9D illustrate possible variations of the tip of the transducer assembly.

FIGS. 10A-10D illustrates one example of use of the device. In the illustrated example, the device creates a collateral channel in the airway wall tissue.

### DETAILED DESCRIPTION OF THE INVENTION

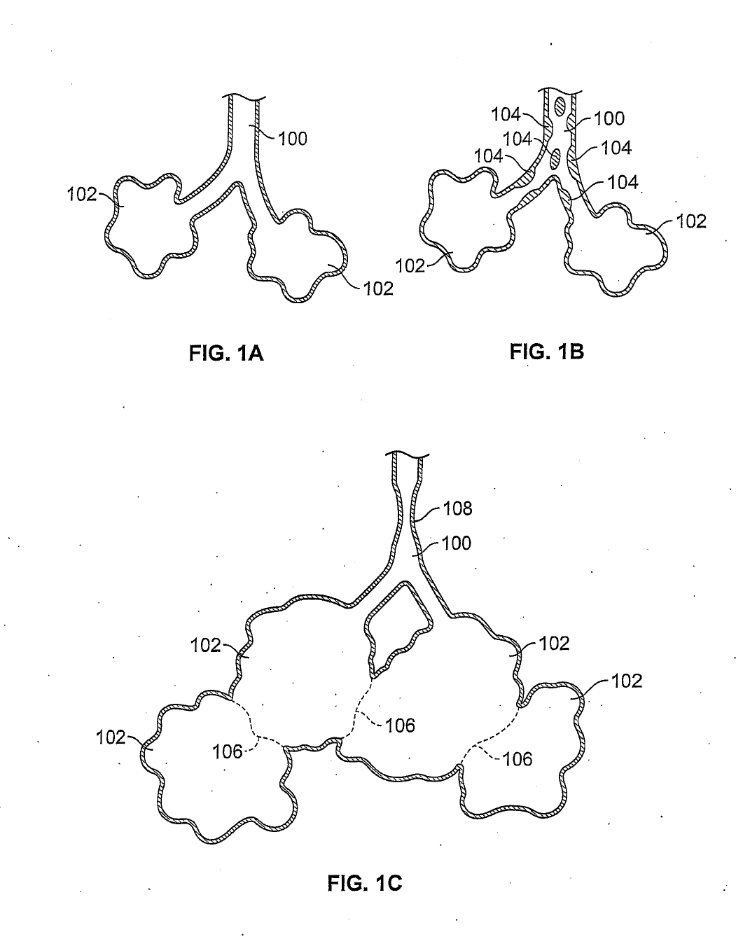

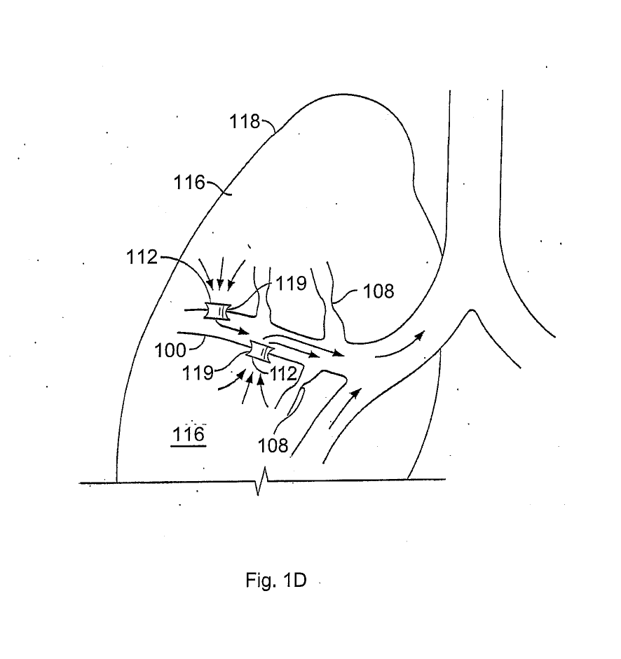

FIG. 1A shows a simplified illustration of a natural airway 100 which eventually branches to a blood gas interface 102. FIG. 1B illustrates an airway 100 and blood gas interface 102 in an individual having COPD. The obstructions 104 (e.g., excessive mucus resulting from COPD, see above) impair the passage of gas between the airways 100 and the interface 102. FIG. 1C illustrates a portion of an emphysematous lung where the blood gas interface 102 expands due to the loss of the interface walls 106 which have deteriorated due to a bio-chemical breakdown of the walls 106. Also depicted is a constriction 108 of the airway 100. It is generally understood that there is usually a combination of the phenomena depicted in FIGS. 1A-1C. More usually, the states of the lung depicted in FIGS. 1B and 1C are often found in the same lung.

As will be explained in greater detail below, the production and maintenance of collateral openings or channels through airway walls permits expired air to pass directly out of the lung tissue and into the airways to ultimately facilitate exchange of oxygen into the blood and/or decompress hyper inflated lungs. The term ‘lung tissue’ is intended to include the tissue involved with gas exchange, including but not limited to, gas exchange membranes, alveolar walls, parenchyma, airway walls and/or other such tissue. To accomplish the exchange of oxygen, the collateral channels allow fluid communication between an airway and lung tissue. Therefore, gaseous flow is improved within the lung by altering or redirecting the gaseous flow within the lung, or entirely within the lung.

FIG. 1D illustrates a schematic of a lung 118 to demonstrate a benefit of the production and maintenance of collateral openings or channels through airway walls. As shown, a collateral channel 112 (located in an airway wall 110) places lung tissue 116 in fluid communication with airways 100 allowing expired air to directly pass out of the airways 100. The term channel is intended to include an opening, cut, slit, tear, puncture, or any other conceivable artificially created opening. As shown, constricted airways 108 may ordinarily prevent air from exiting the lung tissue 116. In the example illustrated in FIG. 1D, there is no implanted structure placed in the collateral channel 112. However, conduits or implants 120 may be placed in the collateral channels 112 to assist in maintaining the patency of the collateral channels 112. Examples of conduits may be found in the applications discussed above. While there is no limit to the number of collateral channels which may be created, it is preferable that 1 or 2 channels are placed per lobe of the lung. For example, the preferred number of channels is 2-12 channels per individual patient. In current trials, it was found that 1-4 channels placed per lobe of the lung and 4-16 channels per individual patient was preferable. This number may vary on a case by case basis. For instance, in some cases an emphysematous lung may require 3 or more collateral channels in one or more lobes of the lung.

The present invention includes the use of a device which is able to detect the presence or absence of a blood vessel by placing a front portion of the device in contact with tissue. One variation of the invention includes the use of Doppler ultrasound to detect the presence of blood vessels within tissue. However, the frequency of the signals is not limited to the ultrasonic range, for example the frequency may be within the range of human hearing, etc.

The ultrasound Doppler operates at any frequency in the ultrasound range but preferably between 2 Mhz-30 Mhz. It is generally known that higher frequencies provide better resolution while lower frequencies offer better penetration of tissue. In the present invention, because location of blood vessels does not require actual imaging, there may be a balance obtained between the need for resolution and for penetration of tissue. Accordingly, an intermediate frequency may be used (e.g., around 8 Mhz). A variation of the invention may include inserting a fluid or gel into the airway to provide a medium for the Doppler sensors to couple to the wall of the airway to detect blood vessels. In those cases where fluid is not inserted, the device may use mucus found within the airway to directly couple the sensor to the wall of the airway.

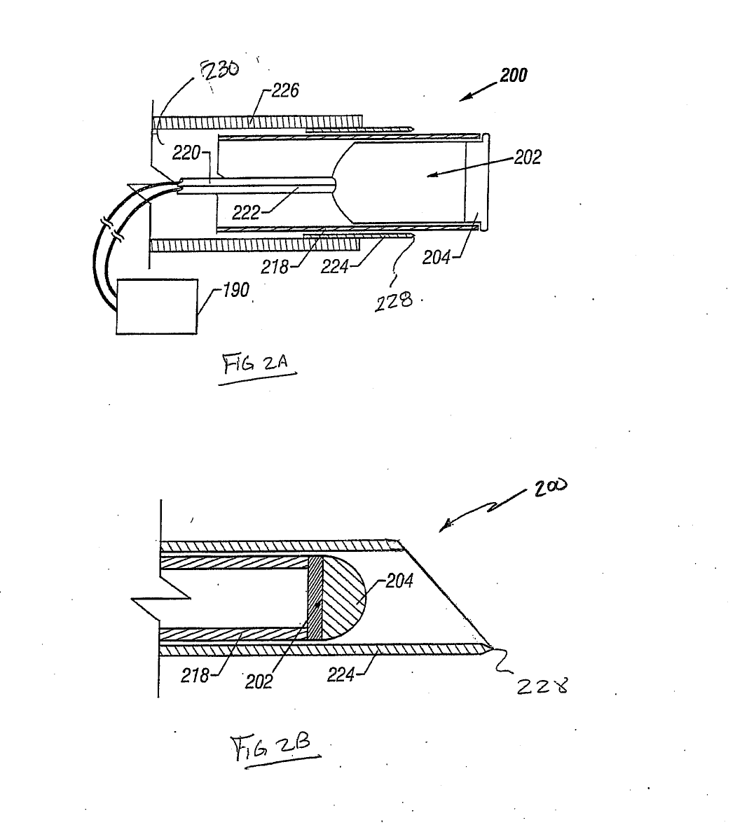

FIG. 2A illustrates a sectional side view of a variation of the inventive device 200. The device 200 includes a transducer assembly 202 (variations of which are described in more detail below.) As shown, a tip 204 is adjacent to the transducer assembly 202 typically the transducer assembly and/or tip will be blunt or atraumatic so that it cannot readily penetrate tissue (e.g., airway tissue, parenchyma, or other such tissue). It is contemplated that, throughout this disclosure, the transducer assembly 202 may be a transducer or a transducer coupled with a covering and other components (examples of which are discussed below). Furthermore, the inventive device 200 may be used without a tip 204 (in which case the transducer (or coated transducer) forms the blunt tip discussed herein). In one variation of the invention, the transducer assembly 202 is located towards a distal portion of an elongate member 218. The transducer assembly of any variation of the present invention may be located within the elongate member, or it may be located within a portion of the tip 204 of the device. In any case, the transducer assembly should be able to move relative to the tissue piercing member 224. The elongate member described herein may be comprised of any commercially available medical-grade flexible tubing. For example, the elongate member may comprise a PTFE material.

The transducer assembly 202 may be coupled to a power supply in any standard manner. For example, the device 200 may include a first conducting member 220 and a second conducting member 222 (e.g., wires) both extending through at least a portion of elongate member 218 to the transducer assembly 202. The conducting members 220, 222 may extend through the lumen of the elongate member 218 or may extend in the wall of the elongate member 218. In any case, the conducting members 220, 222 provide the energy and controls 190 for the transducer assembly 202. For example, the conducting members 220, 222 may be coupled to an ultrasound source 190. Moreover, variations of the inventive device include conducting members 220, 222 which may be comprised of a series of wires, with one set of wires being coupled to respective poles of the transducer, and any number of additional sets of wires extending through the device. Ultimately, the wires enable the device to couple to energy and control units. Although not illustrated, any variation of the device 200 may include an outer sheath (not shown in FIG. 2A) in which the device 200 may be advanced to a target tissue site.

The variation of the device depicted in FIG. 2A includes a tissue piercing member 224 (e.g., a stainless steel thin walled tubing such as a hypo-tube, cannula tubing such as that used for needles, etc.) located at a far end of an elongate member 226. The tissue piercing member 224 includes a sharp tip 228 that is able to penetrate soft tissue or other composite type tissue (e.g., that of an airway wall). The sharp tip described herein will be sharp or have a sufficiently small surface area such that insertion of the tip through tissue may be performed by advancement of the device (or a component thereof). It is contemplated that, where possible, any of the tissue piercing members described herein may be incorporated into any of the variations described herein.

Though the tissue piercing member 224 is shown as being separately attached to the elongate member 226, variations of the invention include elongate members having integral tissue piercing members. In any case, the tissue piercing member 224 may be used to remove or cut tissue (e.g., to create a collateral channel) after the ultrasound assembly 202 locates an acceptable site for creation of the passage.

In the variation depicted in FIG. 2A, the transducer assembly 202 and elongate member 218 are moveable relative to the tissue piercing member 224. Therefore, when the transducer assembly 202 scans the target area for blood vessels, the transducer assembly 202 or tip 204 is distal to or adjacent to the sharp tip 228 (as shown) effectively blunting the sharp tip 228 to prevent undesirable damage to tissue. To create the passage or otherwise remove tissue, the sharp tip 228 is positioned distally to the transducer assembly 202. This latter configuration allows the device to penetrate tissue. In addition, once the device 200 creates the passage, the transducer assembly 202 may then advance into the tissue or passage so that the device 200 can perform additional scans for blood vessels while underneath the surface of the tissue.

FIG. 2A also illustrates the elongate member 226 having a lumen 230, where the lumen 206 and transducer assembly 202 are sized such that there is a close fit therebetween. Additional variations, as described herein, include devices in which the assembly and lumen are sized to permit fluid delivery or suction via the elongate member 226.

FIG. 2B illustrates a variation of a device 200 according to the present invention in which the transducer assembly 202 is placed proximally to the sharp tip of the tissue piercing member 224. In this variation, the tissue piercing member 224 comprises a beveled cannula type structure having a sharp tip 228. In one variation, only the distal most portion of the bevel is a sharp tip 228. In this design, blunting the sharp tip 228 comprises placement of the tip 228 adjacent to the transducer assembly 202.

FIG. 2B also illustrates a transducer assembly 202 provided with a tip 204. It should be noted that the shape of the tips illustrated in the Figs. is not meant to be limiting. Rather, the tip shapes shown are for illustration purposes only and may comprise any atraumatic shape. The transducer assembly 202 shown in FIG. 2B comprises a transducer coupled to an elongate shaft 218. The wires coupling the assembly 202 to a control source are omitted for purposes of illustration.

FIGS. 2C and 2D illustrate variations of the proximal end of variations of the device. FIG. 2C illustrates the transducer assembly 202 and blunt tip 204 within the elongate member 226. The proximal end of the device may be threaded or have an actuation mechanism to allow for placement of the tissue piercing member distal to the blunt tip. FIG. 2D illustrates the device as including an exterior sheath 188. Again, so advancement/retraction mechanism will be located at the proximal end of the device.

It is noted that variations of devices described herein may be constructed to be stiff and inflexible or can be designed to have sufficient flexibility, column strength and length to access the tissue targeted for treatment within tortuous anatomy (such as those devices intended for use in small airways of the lung). Accordingly, for devices used to create collateral channels within lungs, the length of the device should preferably be between 1.5-5 ft long (preferably 4-5 ft) in order to reach the targeted airways.

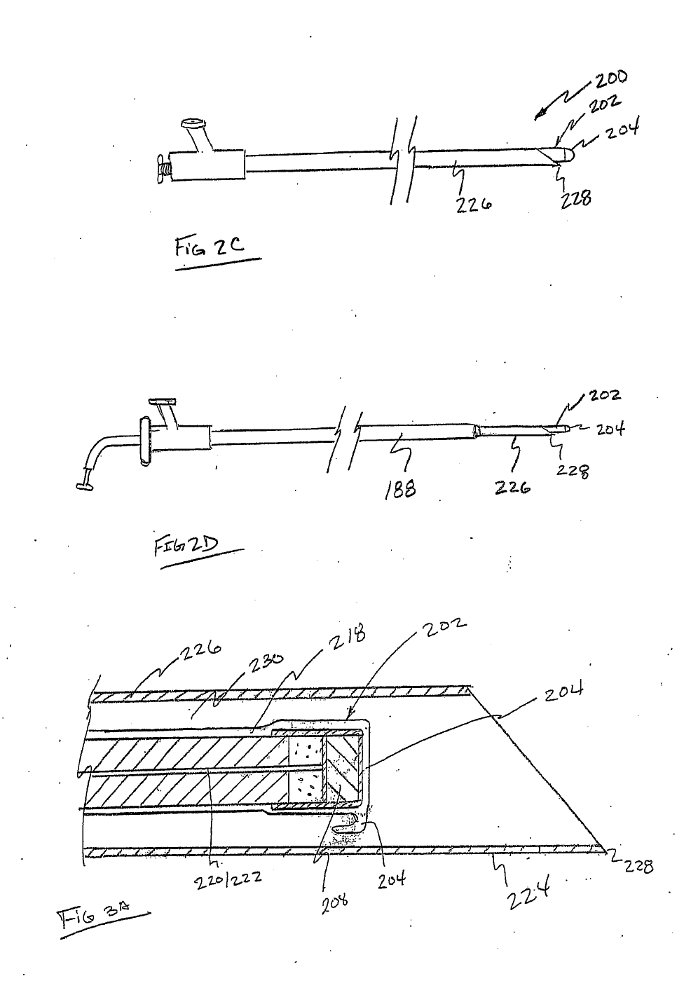

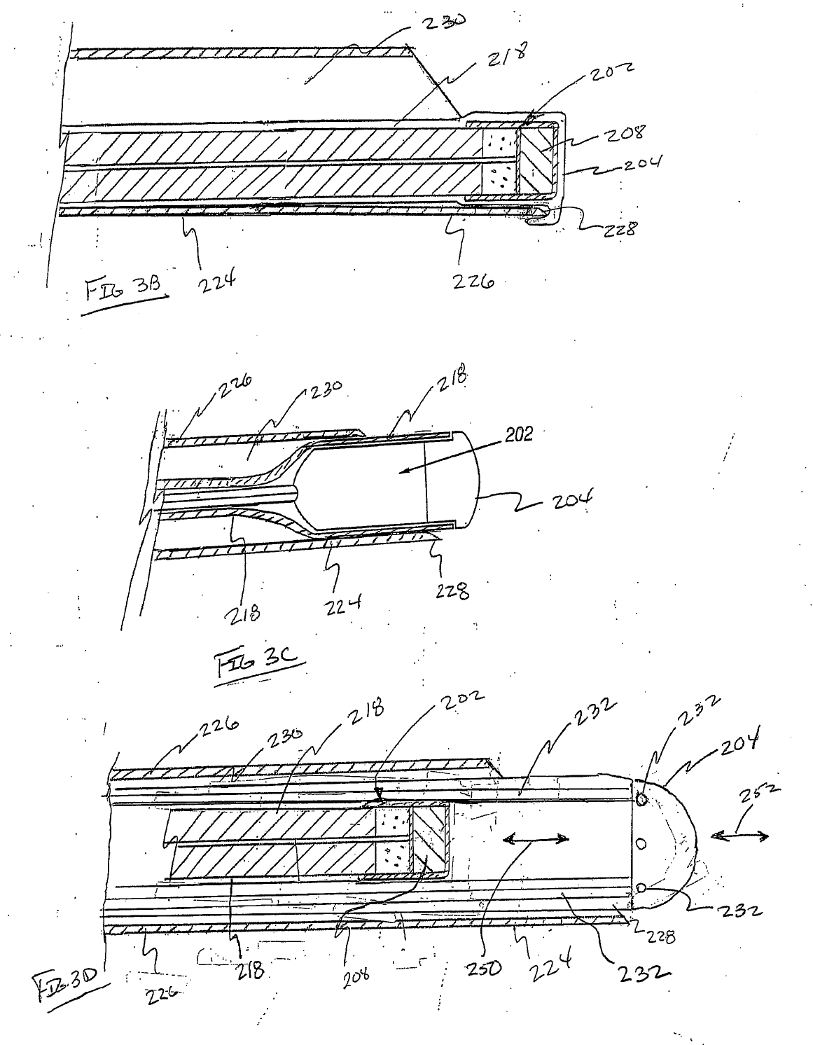

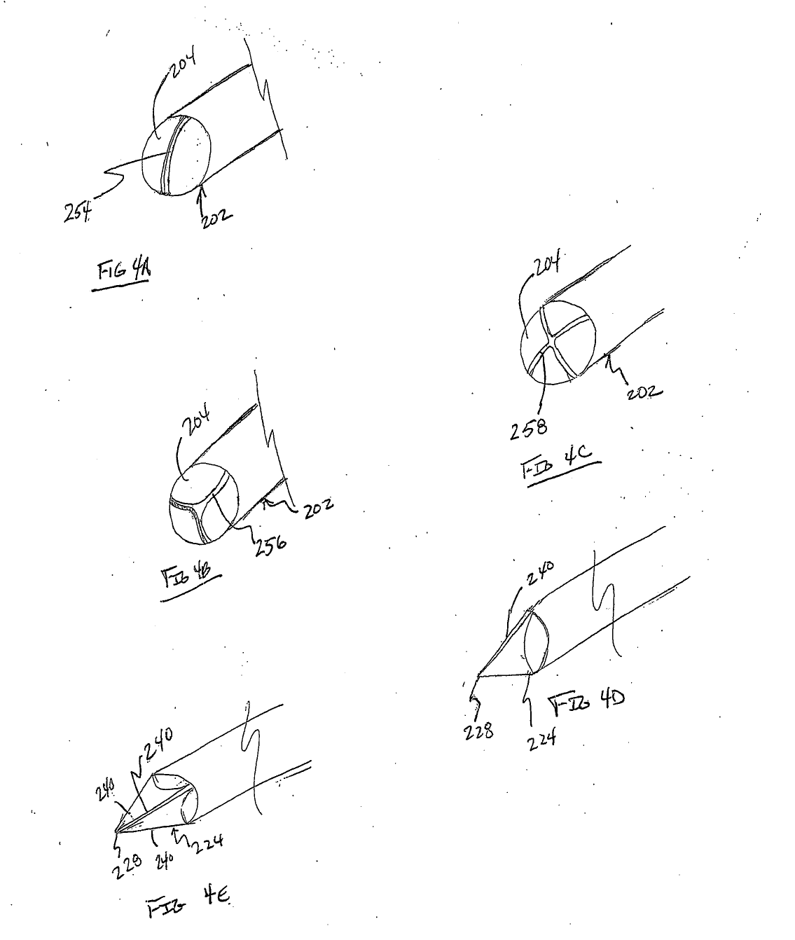

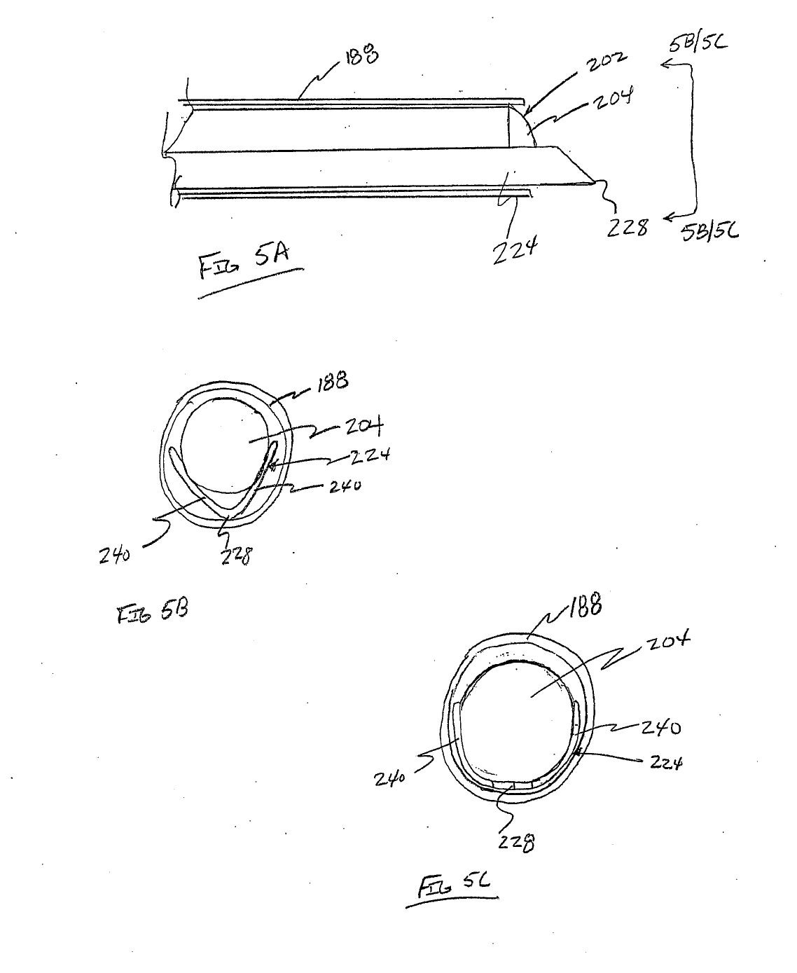

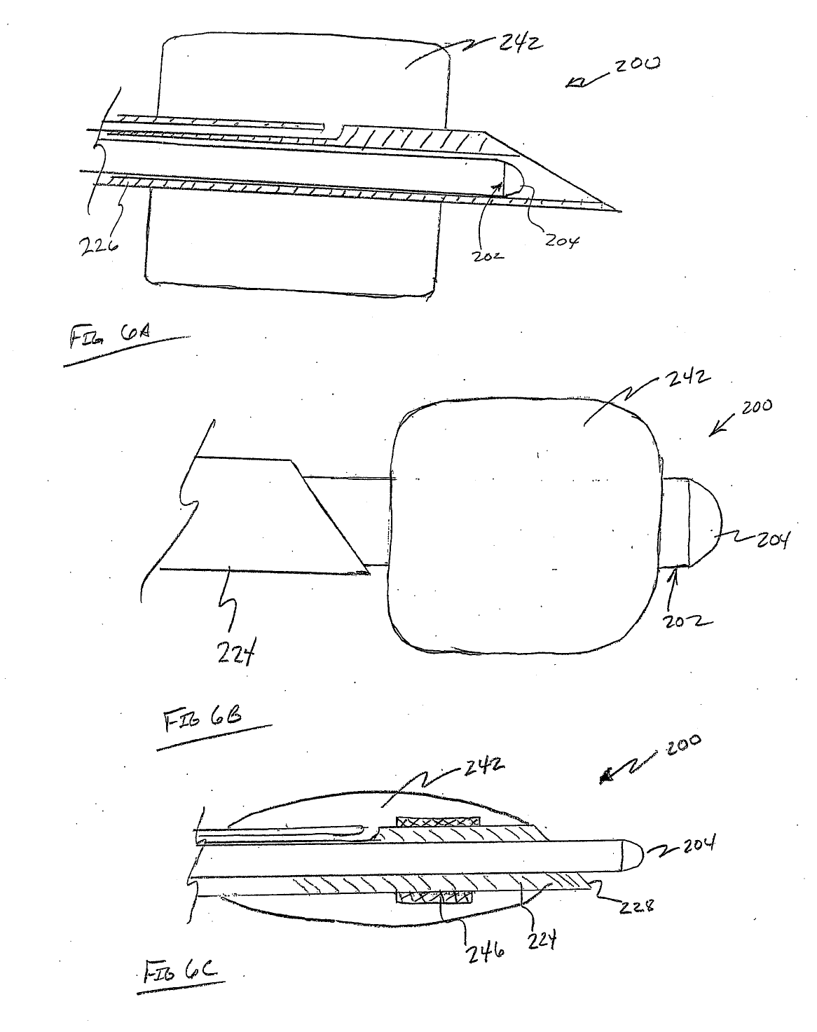

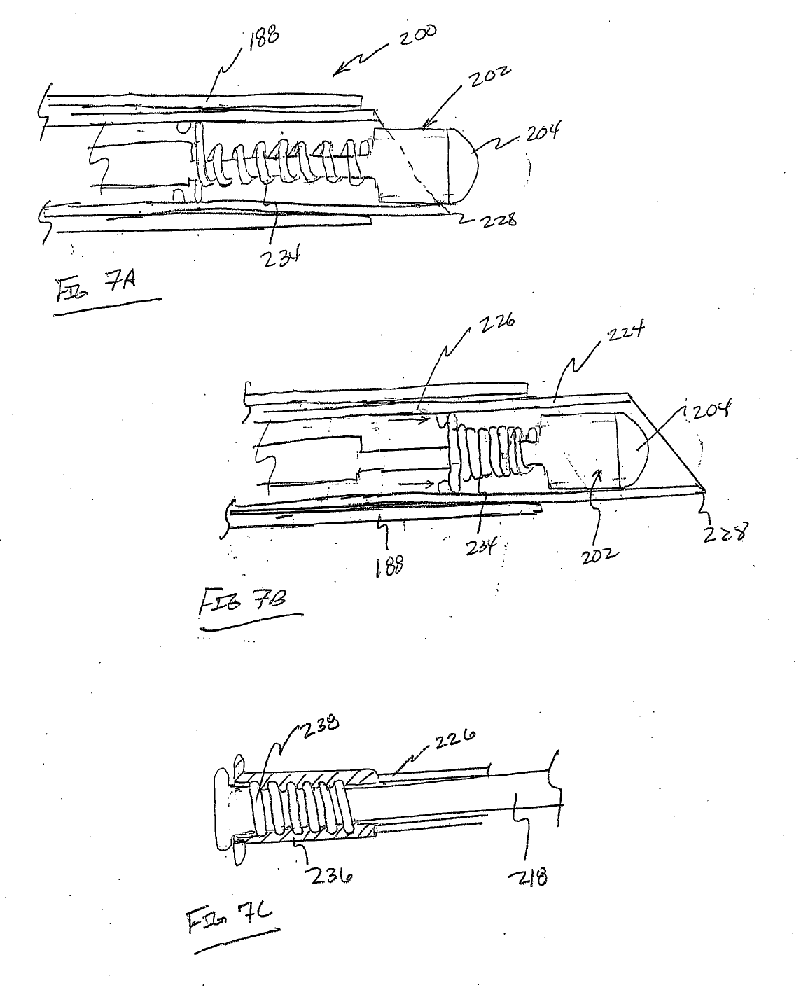

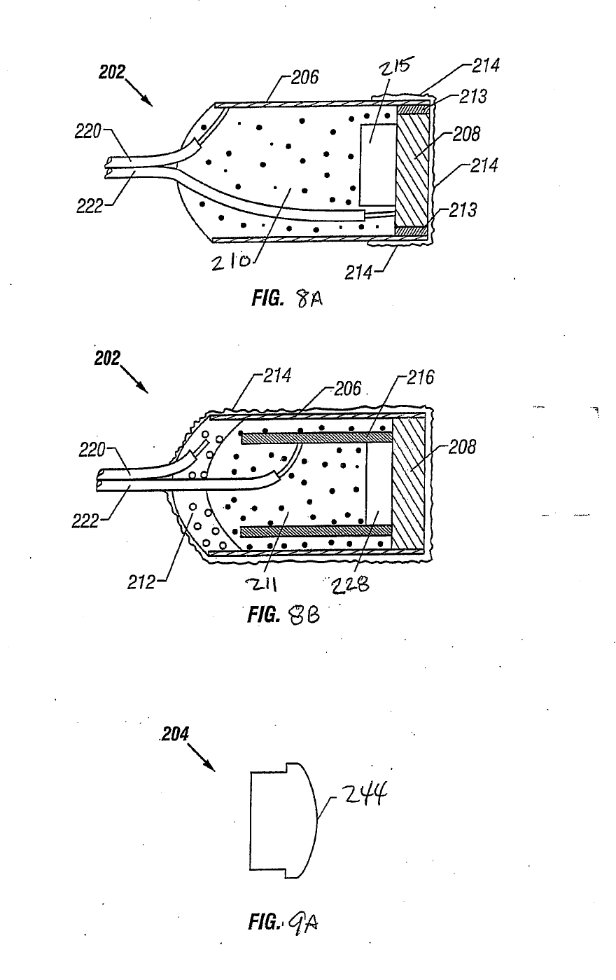

FIG. 3A illustrates another variation of a device 200 according to the present invention. As shown, the device includes a tissue piercing member 226 coupled to a transducer assembly 202. In this variation, the transducer assembly comprises a solid shaft 218 having a backing 210 adjacent to the transducer 208. In this variation, the end of the transducer assembly 202 is a low profile tip (e.g., may be simply a coating over the transducer 208 or a low profile piece attached to the transducer 208).

FIG. 3A also illustrates a variation in which the transducer assembly 202 (and shaft 218) are under-sized relative to the lumen of the elongate member 226. As noted herein, such a configuration permits delivery of fluids or application of suction via the elongate member 226. FIG. 3A also shows a blunt tip 204 in the form of a covering. In this case the covering is attached to the transducer assembly 202 in a manner that allows the covering/blunt tip 204 to nest or seat over the sharp tip 228 of the piercing member 224. This variation may be required to ensure that the blunt tip 204 remains over the sharp tip 228 rather than being deflected within the lumen 230 of the elongate member 226. FIG. 3B illustrates the blunt tip 204 over the sharp tip 228. To assist with manipulation of the cover 204 over the sharp tip 228, the transducer assembly 202 can be rotated as well as axially moveable within the elongate member 226.

FIG. 3C illustrates another variation of a device 200 of the present invention where the transducer assembly 202 is sized to nest within the elongate member 226 and simultaneously blunt the sharpened tip 228. However, the shaft 218 of the transducer assembly 202 is under-sized relative to the lumen 230. Accordingly, to deliver fluid or apply suction, the transducer assembly 202 advances out of the elongate member 226.

FIG. 3D shows another device 200 according to the present invention. In this variation, the blunt tip 204 and transducer assembly 202 are coupled to allow axial movement of the transducer assembly within the blunt tip 204 (as shown by arrows 250). In some variations, the transducer assembly 204 is removable from the blunt tip 204 and even removable from the device 200.

FIG. 3D also illustrates additional features of the blunt tip 204. As shown, the blunt tip may include ports 232 for fluid delivery or suction. The blunt tip 204 is also coupled to the elongate member 226 to permit relative movement therebetween (as shown by arrows 252).

FIGS. 4A-4E illustrate variations of devices according to the present invention where ultrasound transducer assembly 202 is coupled to the sharp tip 228 in a manner that allows movement of the tissue piercing member 224 through the transducer assembly 202. As illustrated the transducer assembly 202 may be sectioned to form an “I”, “Y”, or “X” opening 254, 256, and 258 in FIGS. 4A, 4B, 4C respectively. Each device shall include a sharp tip having a configuration that matches the shape of the respective opening. As shown in FIGS. 4D-4E, the corresponding tissue piercing member 224 will have a sharp tip 228 and an edge 240. Upon penetration of the tissue by the sharp tip 228, the edge 240 creates a slit or expands the opening of the tissue. It is contemplated that this listing is non-exhaustive and that varying shaped openings are within the scope of the invention.

FIGS. 5A-5C illustrate another variation of the device 200 where the tissue piercing member 224 and/or elongate member 226 only partially cover the transducer assembly 202. FIG. 5A illustrates partial cross sectional view of a sheath 188 carrying the transducer assembly 202 and tissue piercing member 224. In this variation the tissue piercing member 224 comprises a “V” or “U” shaped cross section. It is noted that the elongate member 226 may also comprise the same cross section or, alternatively, may have a circular (or similar) shaped cross section. FIGS. 5B and 5C show alternate front views of variations of the invention taken along the lines 5B-5B and 5C-5C of FIG. 5A. The shape of the tissue piercing member 224 may be “V” or “U” shaped as shown in FIG. 5B and 5C respectively. In either case, the tissue piercing member 224 includes a sharp tip 228 along with one or more sharp edges 240 that slit or open the tissue.

FIGS. 6A and 6B illustrate additional aspects of the invention that may be incorporated into the device described herein. As shown, in FIG. 6A, the device 200 may include an expandable member such as a balloon 242 on the exterior of the elongate member 226. Alternatively, or in combination, as shown in FIG. 6B, a balloon 242 may be located on the shaft of the transducer assembly 218. It should be noted that other mechanical type expandable members may be used in place of, or in addition to balloons.

The use of a balloon 242 allows dilation of the passage in tissue created by the tissue piercing member 224. Variations of the invention can be designed for use in tough tissue that is resistant to radial expansion (such as an airway wall). In such variations, the balloon may comprise non-distensible balloon to overcome the toughness of the tissue . Non-distensible balloons are generally made up of relatively inelastic materials consisting of PET, nylons, polyurethanes, polyolefins, PVC, and other crosslinked polymers. Therefore, use of a non-distensible balloon allows for easier expansion of tissue because the non-distensible balloon permits high pressurization (>6 atm). Moreover, non-distensible balloons generally inflate in a uniform shape since the balloon unfolds to assume an expanded shape. In contrast, distensible balloons typically expand in shape when pressurized. In any case, it should be noted that distensible and/or non-distensible balloons may be used in the present invention depending upon the application.

Non-distensible balloons typically occupy a greater mass than distensible balloons because the non-distensible balloon is inelastic and is folded in an unexpanded shape. Therefore, variations of the invention include non-distensible balloons having a working diameter (or diameter in an unexpanded shape) that is close to the diameter of the piercing member. This allows insertion of the unexpanded balloon into the opening created by the piercing member. Accordingly, balloons of the present invention may include thin walled balloons, balloons with small distal profiles, balloons with distal ends that are close in actual diameter to the diameter of the piercing member, or balloons that folds into low profile state, or balloons having a combination of these features.

FIG. 6C illustrates an additional variation of the device 200 that includes a second transducer 246 (such as a ring transducer) located within the balloon 242. In this variation, when the balloon expands to dilate a passage, the second transducer permits the balloon 242 to perform additional scans for blood vessels.

FIG. 7A illustrates a variation of the device 200 having a spring member 234. The spring member 234 can provide a safety feature by biasing the sharp tip 228 proximally to the blunt tip 204. After a medical practitioner confirms a target site is free from any blood vessels, the practitioner may apply a force to compress the spring, moving the sharp tip 228 distally to the blunt tip 204 (as illustrated in FIG. 7B). In such a case, the transducer assembly 202 will be fixed relative to the outer sheath 188. Variations of the invention include configuring the device to allow movement of the transducer assembly proximally to the tissue piercing member. However, movement of the tissue piercing member provides a safety measure as the tissue piercing member will naturally be “blunted”. It is noted that the spring member 234 may be placed anywhere along the length of the device. Furthermore, although not shown, the spring member 234 can be placed between the elongate member 226 and the sheath 188.

FIG. 7C shows another variation of the invention having a first and second threaded portion 236, 238 located in hubs affixed to the near end of the respective elongate member 226 and shaft 218. The threaded portion allows for axial advancement and retraction of the blunt tip relative to the sharp tip. Such a feature is useful to control advancement of either tip. As noted above, the threaded portions 236, 238 may be located anywhere along the length of the device.

As discussed herein, for some variations of the invention it is desirable to minimize the size of the device especially at the distal end. Although the invention may be any size, it was found that an overall device diameter of 0.071″ was acceptable.

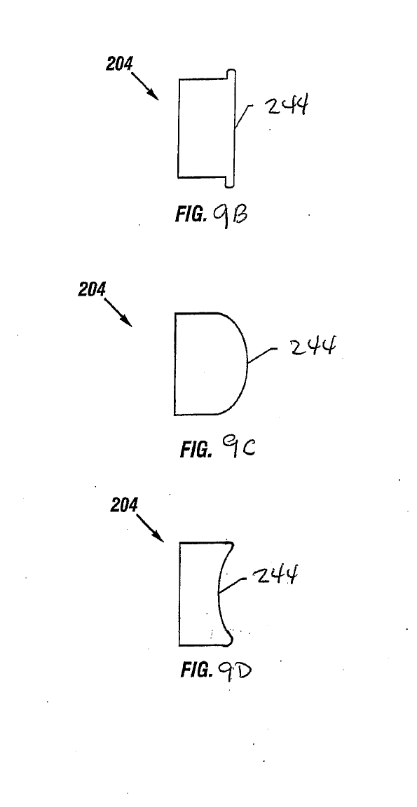

FIGS. 8A-8B illustrate a non-exhaustive sample of variations of the transducer assembly 202 configured to reduce an overall size of the assembly. It is noted that the invention may use any type of transducer assembly. FIG. 8A illustrates a cross-sectional view of a basic variation of a transducer assembly 202. For illustration purposes, the transducer assembly 202 illustrated in FIG. 8A is shown without a tip. The transducer assembly 202 includes at least one transducer 208 (e.g., a piezoelectric transducer.) In this variation, the front surface of the transducer 208 comprises a first pole and the rear surface comprises a second pole.

The transducer or transducers may comprise a piezo-ceramic crystal (e.g., a Motorola PZT 3203 HD ceramic). In the current invention, a single-crystal piezo (SCP) is preferred, but the invention does not exclude the use of other types of ferroelectric material such as poly-crystalline ceramic piezos, polymer piezos, or polymer composites. The substrate, typically made from piezoelectric single crystals (SCP) or ceramics such as PZT, PLZT, PMN, PMN-PT; also, the crystal may be a multi layer composite of a ceramic piezoelectric material. Piezoelectric polymers such as PVDF may also be used. Micromachined transducers, such as those constructed on the surface of a silicon wafer are also contemplated (e.g., such as those provided by Sensant of San Leandro, CA.) As described herein, the transducer or transducers used may be ceramic pieces coated with a conductive coating, such as gold. Other conductive coatings include sputtered metal, metals, or alloys, such as a member of the Platinum Group of the Periodic Table (Ru, Rh, Pd, Re, Os, Ir, and Pt) or gold. Titanium (Ti) is also especially suitable. The transducer may be further coated with a biocompatible layer such as Parylene or Parylene C.

The covering 206 of the transducer assembly 202 may contain at least a portion of the transducer 208. In some variations of the invention, the covering 206 may comprise a conductive material. In such cases the covering 206 itself becomes part of the electrical path to the first pole of the transducer 208. Use of a conductive covering 206 may require insulating material 213 between the sides of the transducer 208, thereby permitting a first conductive medium 214 to electrically couple only one pole of the transducer 208 to the covering 206.

At least a portion of the front surface of the transducer 208 will be in contact with the conductive medium 214. The conductive medium 214 permits one of the poles of the transducer 208 to be placed in communication with a conducting member that is ultimately coupled to a power supply. As shown in this example, the conductive medium 214 places the pole of the transducer 208 in electrical communication with the covering 206. In some variations the conductive medium 214 may coat the entire transducer 208 and covering 206. Alternatively, the conductive medium 214 may be placed over an area small enough to allow for an electrical path between a conducting member and the respective pole of the transducer 208. The conductive medium 214 may be any conductive material (e.g., gold, silver, tantalum, copper, chrome, or any bio-compatible conductive material, etc. The material may be coated, deposited, plated, painted, wound, wrapped (e.g., a conductive foil), etc. onto the transducer assembly 202.

The transducer assembly 202 depicted in FIG. 8A also illustrates conducting members 220, 222 electrically coupled to respective poles of the transducer 208. Optionally, the conducting members 220, 222 may be encapsulated within an epoxy 211 located within the covering 206. The epoxy 211 may extend to the transducer 208 thereby assisting in retaining both the conducting members 220, 222 and transducer 208 within the covering. It may also be desirable to maintain a gap 228 between the transducer 208 and any other structure. It is believed that this gap 228 improves the ability of the transducer assembly 202 to generate a signal.

FIG. 8B illustrates another variation of a transducer assembly 202. In this variation, the conductive medium 214 extends over the entire transducer covering 206. Accordingly, the covering 206 may be made of a non-conducting material (e.g., a polyamide tube, polyetherimide, polycarbonate, etc.) The transducer assembly 202 may further comprise a second tube 216 within the covering 206. This second tube 216 may be a hypo-tube and may optionally be used to electrically couple one of the conducting members to a pole of the transducer 208. As shown, the covering 206 may contain a non-conductive epoxy 210 (e.g., Hysol 2039/3561 with Scotchlite glass microspheres B23/500) which secures both the conducting member and the second tube 216 within the covering 206. This construction may have the further effect of structurally securing the transducer 208 within the assembly 202. Again, a gap 228 may or may not be adjacent to the transducer to permit displacement of the transducer 208.

FIG. 8B also illustrates the assembly 202 as having a conductive epoxy 212 which encapsulates the alternate conducting member 220. An example of a conductive epoxy is Bisphenol epoxy resin with silver particulates to enable conductivity. The particulates may be from 70-90% of the resin composition. The resin may then be combined with a hardener (e.g., 100 parts resin per 6 parts hardener.) The conductive epoxy 212 is in electrical communication with the conductive medium 214 allowing for a conductive path from the conducting member 220 to the conductive medium 214. Accordingly, use of the conductive epoxy 212 secures the conducting member 220 to the assembly 202 while electrically coupling the conducting member 220 to the transducer via the conductive coating 214.

Although variations of the transducer assembly include a tip and housing, the invention may omit the transducer covering and other structures not necessary to generate a source signal and receive a reflected signal. Therefore, it is contemplated that the invention may simply have a transducer that is coupled to a controller.

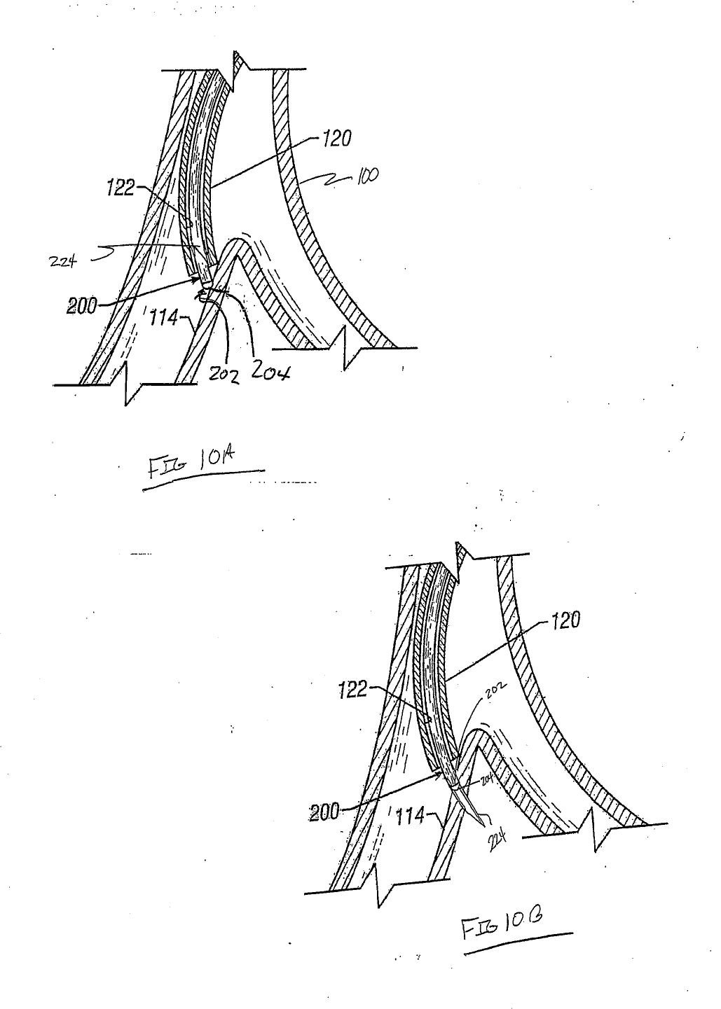

FIGS. 9A-9D, illustrate possible variations of the tip 204 of the transducer assembly. It is noted that these variations are provided for illustrative purposes and are not meant to be exhaustive. The tips 204 of the present invention may function simply as a blunting tip (but still passes and receives ultrasound signals) or as a lens to disperse and/or direct the signal over a substantial portion of the outer surface of the tip 204. When configured to function as a lens, the tip 204 is adapted to disperse and/or direct (e.g., by diffraction) a reflected signal towards the transducer (not shown in FIGS. 9A-9D). Accordingly, given the above described configuration, the inventive device 200 will be able to detect vessels with substantially most of the tip 204. The tip may comprise a signal directing means.

When configured to function as a lens, the tip 204 is designed such that it interferes and redirects the signals in a desired direction in a manner like a lens. It also may be desirable to place an epoxy between the tip 204 and the transducer. Preferably, the epoxy is thin and applied without air gaps, bubbles or pockets. Also, the density/hardness of the epoxy should provide for transmission of the signal while minimizing any effect or change to the source signal. The configuration of the transducer assembly 202 permits the lens tip 204 to disperse a signal over a substantial portion of its outer surface 244. The lens tip 204 also is adapted to refract a reflected signal towards the transducer 208. Accordingly, given the above described configuration, the inventive device will be able to detect vessels with any part or substantially all of the lens tip 204 that contacts tissue.

Although the tip of the present invention is able to transmit a source signal and receive a reflected signal, the invention is not limited to requiring both functions. For example, the inventive device could be configured to generate a source signal and direct the source signal to an area of interest but a second device or transducer assembly could be used to receive the reflected signal. Accordingly, a separate device could be used to generate the source signal with the inventive device being used to receive the reflected signal.

The tip 204 may be comprised of materials such as a dimethyl pentene, a methylpentene copolymer (plastic-TPX), aluminum, carbon aerogel, polycarbonate (e.g., Lexan), polystyrene, or etc., any standard material used for ultrasound applications.

As illustrated in FIG. 9A, although the front surface 244 of the tip 202 is illustrated as being hemispherical, the tip 204 may have other profiles as well. For example, it is desirable that the tip 204 produce a certain amount of divergence of the signal being passed therethrough. However, depending on a variety of factors (e.g., material, frequency of the signal, etc.) a tip 204 may encounter excessive divergence which is destructive to the outgoing signal. Accordingly, it may be desirable to produce a tip 204 as illustrated in FIG. 9B in which a front surface 244 of the tip 204 is substantially flat. The degree of flatness of the tip 204 will often depend upon experimentation to reduce the amount of destructive reflections, thus minimizing excessive divergence due to differences in speed of sound in tip versus tissue. For example, when using a tip that is conducive to an ultrasound signal (e.g., TPX) a rounded tip can be used since there is not excessive divergence of the source signal. Use of a material that is not as conducive to ultrasound requires a flatter tip due to the resulting divergence of the source signal. FIG. 9C illustrates another variation of a tip 204 having a rounded front surface 244 but with no projections on the sides of the tip 204. FIG. 9D illustrates a tip 204 with a concave front surface 244.

In any case, the tip will be configured to avoid sharp edges that may cause any unintended damage to tissue while the device is being used to determine the presence or absence of a blood vessel. In such a case, for example, the tip may be designed such that it doesn't have sharp edges, or any sharp edges may be covered by other parts of the device (e.g., the elongate member, an outer sheath, etc.)

Commonly assigned patent publication nos. US20020128647A1; US20020138074A1; US20030130657A1, and US20050107783A1; disclose additional variations of transducer assemblies and modes of securing such assemblies to the device. The entirety of each of which is incorporated by reference herein.

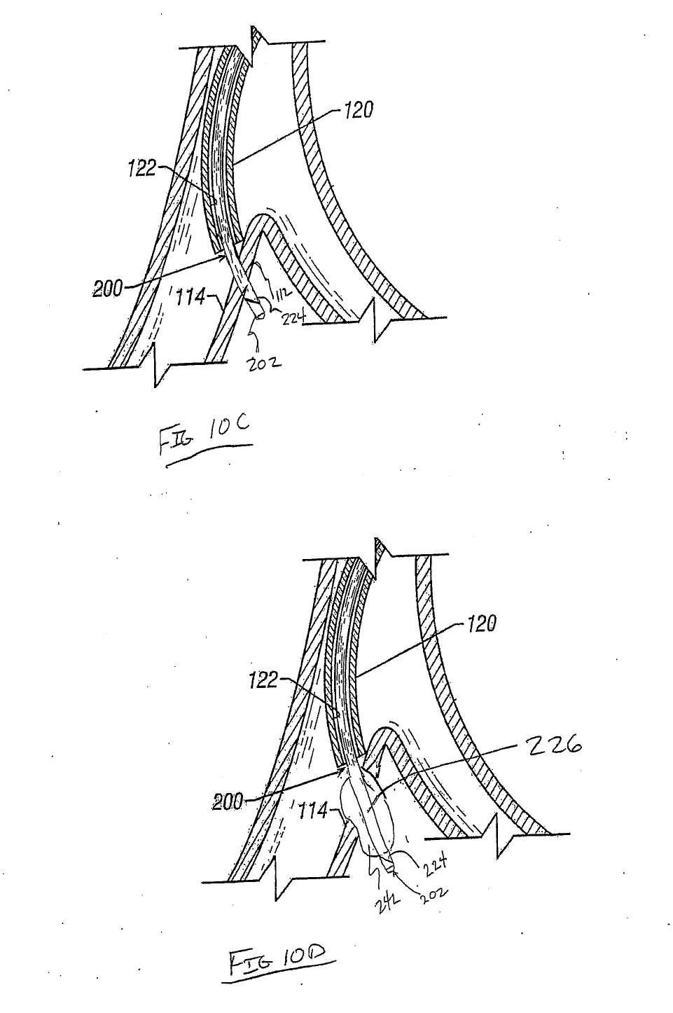

FIG. 10A-10C illustrates one example of use of the device. In the illustrated example, the device creates a collateral channel in the airway wall tissue within a lung. However, it is understood that the device may be used in any part of the body and for any application. For example, variations of the device may be used during a biopsy procedure to scan for blood vessels, and remove a biopsy sample within the tissue piercing member.

FIG. 10A illustrates optional use of an access device 119 advanced into the airways 100 of a lung. The access device may be a bronchoscope, endoscope, endotracheal tube with or without vision capability, or any type of delivery device. The access device 120 will have at least one lumen or working channel 122. The access device 120 will locate an approximate site 114 for creation of a collateral channel. For example, location of the site may be accomplished visually, or with additional equipment such as a CT scan to locate areas for treatment. In cases where the access device 120 is a bronchoscope or similar device, the access device 120 is equipped so that the surgeon may observe the site for creation of the collateral channel. In some cases it may be desirable for non-invasive imaging of the procedure. In such cases, the access device 120 as well as the other devices discussed herein, may be configured for detection by the particular non-invasive imaging technique such as fluoroscopy, “real-time” computed tomography scanning, or other technique being used.

FIG. 10A also illustrates advancement of a variation of the inventive device 200 through the channel 122 of the access device 120 towards the target site 114. The medical practitioner then uses the transducer assembly 202 to inspect the target site to determine whether a blood vessel is adjacent to the site. If a blood vessel is detected, then another target site may be selected. As illustrated, the tissue piercing member 224 is proximal to the blunt tip 204. Such an arrangement minimizes the risk of creating unwanted damage to tissue. As shown, the tissue piercing member 224 may be withdrawn into the access device 120. Alternatively, the tissue piercing member 224 may be placed outside of the access device, but will be blunted by the blunt tip 204.

FIG. 10B illustrates the device 200 after the tissue piercing member 224 is placed distally to the transducer assembly. The tissue piercing member 224 then creates a collateral channel. It is noted that either the access device 120 or the inventive device 200 may be steerable. Such a feature may assist in the positioning of any of the devices used in the inventive method. Although it is not illustrated, as discussed herein, it is desirable to create the collateral channel such that it is in fluid communication with an air-sac. The fluid communication allows for the release of trapped gasses from the hyper-inflated lung.

FIG. 10C illustrates use of the device 200 to perform an additional scan for adjacent blood vessels. As shown, the device 200 can be inserted through the newly created passage to perform a scan for blood vessels underneath the surface of the tissue or within the opening of the passage. As shown, the blunt tip 204 may be positioned to minimize the chance that the tissue piercing member 224 causes undesired damage to tissue. At any point, saline, other fluids or other substances may be inserted into and/or around the opening to assist in scanning the tissue.

FIG. 10D illustrates another step in which the opening is dilated by an expandable member 242. In this variation, the balloon 242 is located on the elongate member 226. As noted herein, in some variations of the invention, the balloon 242 is equipped with a second transducer. This configuration allows for additional scanning for blood vessels.

After dilation of the passage, the device may be removed. Alternatively, the expanded passage may be filled with fluid for additional scanning via the transducer assembly.

A further variation of the invention may include configuring the transducer assembly and/or controller to have different levels of sensitivity. For example, a first level of sensitivity may be used to scan the surface of tissue. Then, after creation of the opening, the second level of sensitivity may be triggered. Such a feature acknowledges that scanning of tissue on, for example, the airway wall may require a different sensitivity than when scanning tissue within the parenchyma of the lung.

It should be noted that the invention includes kits containing the inventive device with any one or more of the following components, a Doppler ultrasound controller, a conduit as described in one or more of the applications listed above, and a bronchoscope/endoscope.

In the above explanation of Figs., similar numerals may represent similar features for the different variations of the invention.

The invention herein is described by examples and a desired way of practicing the invention is described. However, the invention as claimed herein is not limited to that specific description in any manner. Equivalence-to the description as hereinafter claimed is considered to be within the scope of protection of this patent.

The devices of the present invention are configured to locate a target site for creation of a collateral channel in the tissue and to create an opening in tissue. As discussed above, a benefit of this combination feature is that a single device is able to select a target location and then create an opening without having been moved. Although the device is discussed as being primarily used in the lungs, the device is not limited as such and it is contemplated that the invention has utility in other areas as well, specifically in applications in which blood vessels or other structures must be avoided while cutting or removing tissue (one such example is tumor removal).

The above illustrations are examples of the invention described herein. It is contemplated that combinations of aspects of specific embodiments/variations or combinations of the specific embodiments/variations themselves are within the scope of this disclosure.

We claim:

1. A medical device for creating passages in tissue and sensing blood vessels in or around the passages, the device comprising:
an elongate member having a near end and a far end, the far end including a tissue piercing member having a sharp tip able to penetrate soft tissue; and
an ultrasound transducer assembly coupled to the tissue piercing member such that the ultrasound transducer assembly may axially move relative to the tissue piercing member; and
a blunt tip coupled to the ultrasound transducer assembly such that when the blunt tip is moved at least adjacent to or distal to the sharp tip, the tissue piercing member is unable to penetrate soft tissue.

2. The medical device of claim 1, where the elongate member further comprises at least one lumen extending at least partially therethrough.

3. The medical device of claim 2, where the ultrasound transducer assembly is sized relative to the lumen in the elongate member to form a close tolerance sufficient for a sliding fit therebetween.

4. The medical device of claim 2, Where the ultrasound transducer is sized smaller than a size of the lumen in the elongate member such that the elongate member lumen enables delivery of fluid or application of suction to the tissue.

5. The medical device of claim 1, where the tissue piercing member further comprises a sharp edge, such that upon penetration of soft tissue by the sharp tip, the sharp edge creates a slit in tissue.

6. The medical device of claim 1, further comprising an expandable member on an exterior surface of the elongate member and adjacent to the far end.

7. The medical device of claim 6, where the expandable member comprises a balloon.

8. The medical device of claim 7, where the balloon is a non-distensible balloon.

9. The medical device of claim 7, where the balloon is a distensible balloon.

10. The medical device of claim 7, further comprising a second ultrasound transducer assembly within the balloon.

11. The medical device of claim 1, further comprising a shaft attached to the transducer assembly.

12. The medical device of claim 11, where the shaft includes an expandable member located proximal to the tissue piercing member.

13. The medical device of claim 12, where the expandable member comprises a balloon.

14. The medical device of claim 13, where the balloon is a non-distensible balloon.

15. The medical device of claim 13, where the balloon is a distensible balloon.

16. The medical device of claim 11, further comprising a spring member coupled to both the shaft and the elongate member such that in a relaxed state, the spring causes the blunt tip distally to be distal to the sharp tip, and compression of the spring member causes the sharp tip to be distal to the blunt tip.

17. The medical device of claim 11, where the elongate member comprises a first threaded portion and the ultrasound transducer assembly shaft comprises a second threaded portion, such that rotation of the shaft relative to the elongate member causes axial movement of the transducer assembly.

18. The medical device of claim 1, where the blunt tip comprises a front face of the ultrasound transducer assembly.

19. The medical device of claim 19, where the blunt tip comprises a shape selected from the group consisting of a rounded shape, a flat shape, and a convex shape.

20. The medical device of claim 1, comprising at least one port opening located in the blunt tip, where the port opening enables delivery of fluid or application of suction to the tissue.

21. The medical device of claim 1, where the tissue piercing member comprise a sharpened cannula.

22. The medical device of claim 1, where the tissue piercing member comprise a beveled needle-type cannula.

23. The medical device of claim 1, where the tissue piercing member comprises a cross-sectional shape selected from a V-shape or a U-shape.

24. The medical device of claim 1, where the ultrasound transducer assembly is moveably located within the tissue piercing member.

25. The medical device of claim 1, where the ultrasound transducer assembly is moveably located adjacent to the tissue piercing member.

26. The medical device of claim 1, where the the tissue piercing member is moveably located adjacent to ultrasound transducer assembly.

27. The medical device of claim 1, where the ultrasound transducer assembly comprises a plurality of ultrasound transducers.

28. The medical device of claim 27, where the ultrasound transducer assembly is affixed to a shaft and where each ultrasound transducer is joined to an end of the shaft and spaced from an adjacent ultrasound transducer by a gap.

29. The medical device of claim 28, where the tissue piercing member is slidable through the gap of the transducer assembly.

30. The medical device of claim 29, where the gap comprises a shape selected from an I shape, Y shape, and X shape.

31. The medical device of claim 30, where the tissue piercing member includes a sharp edge, where the sharp edge forms an I shape, Y shape, and X shape.

32. A medical device for creating passages in tissue and sensing blood vessels in or around the passages, the device comprising:
an elongate member having a near end and a far end, the far end including a tissue piercing member having a sharp tip able to penetrate soft tissue; and
an ultrasound transducer assembly coupled to the tissue piercing member such that the ultrasound transducer assembly may axially move relative to the tissue piercing member; and
an expandable member adapted to dilate the passage.

33. The medical device of claim 32, further comprising a blunt tip coupled to the ultrasound transducer assembly such that when the blunt tip is moved at least adjacent to or distal to the sharp tip, the tissue piercing member is unable to penetrate soft tissue.

34. The medical device of claim 32, where the elongate member further comprises at least one lumen extending at least partially therethrough.

35. The medical device of claim 34, where the ultrasound transducer assembly is sized relative to the lumen in the elongate member to form a close tolerance sufficient for a sliding fit therebetween.

36. The medical device of claim 34, where the ultrasound transducer is sized smaller than a size of the lumen in the elongate member such that the elongate member lumen enables delivery of fluid or application of suction to the tissue.

37. The medical device of claim 32, where the tissue piercing member further comprises a sharp edge, such that upon penetration of soft tissue by the sharp tip, the sharp edge creates a slit in tissue.

38. The medical device of claim 32, where the expandable member is located on an exterior surface of the elongate member and adjacent to the far end.

39. The medical device of claim 38, where the expandable member comprises a balloon.

40. The medical device of claim 39, where the balloon is a non-distensible balloon.

41. The medical device of claim 39, where the balloon is a distensible balloon.

42. The medical device of claim 39, further comprising a second ultrasound transducer assembly within the balloon.

43. The medical device of claim 32, further comprising a shaft attached to the transducer assembly and where the expandable member is located on the shaft and proximal to the tissue piercing member.

44. The medical device of claim 43, where the expandable member comprises a balloon.

45. The medical device of claim 44, where the balloon is a non-distensible balloon.

46. The medical device of claim 44, where the balloon is a distensible balloon.

47. The medical device of claim 32, where the elongate member comprises a first threaded portion and the ultrasound transducer assembly shaft comprises a second threaded portion, such that rotation of the shaft relative to the elongate member causes axial movement of the transducer assembly.

48. The medical device of claim 32, where the blunt tip comprises a front face of the ultrasound transducer assembly.

49. The medical device of claim 48, where the blunt tip comprises a shape selected from the group consisting of a rounded shape, a flat shape, and a convex shape.

50. The medical device of claim 32, comprising at least one port opening located in the blunt tip, where the port opening enables delivery of fluid or application of suction to the tissue.

51. The medical device of claim 32, where the tissue piercing member comprise a sharpened cannula.

52. The medical device of claim 32, where the tissue piercing member comprise a beveled needle-type cannula.

53. The medical device of claim 32, where the tissue piercing member comprises a cross-sectional shape selected from a V-shape or a U-shape.

54. The medical device of claim 32, where the ultrasound transducer assembly is moveably located within the tissue piercing member.

55. The medical device of claim 32, where the ultrasound transducer assembly is moveably located adjacent to the tissue piercing member.

56. The medical device of claim 32, where the the tissue piercing member is moveably located adjacent to ultrasound transducer assembly.

57. The medical device of claim 32, where the ultrasound transducer assembly comprises a plurality of ultrasound transducers.

58. The medical device of claim 57 where the ultrasound transducer assembly is affixed to a shaft and where each ultrasound transducer is joined to an end of the shaft and spaced from an adjacent ultrasound transducer by a gap.

59. The medical device of claim 58, where the tissue piercing member is slidable through the gap of the transducer assembly.

60. The medical device of claim 59, where the gap comprises a shape selected from an I shape, Y shape, and X shape.

61. The medical device of claim 60, where the tissue piercing member includes a sharp edge, where the sharp edge forms an I shape, Y shape, and X shape.

62. A method of treating lung tissue, method comprising:
selecting an area in lung tissue;
examining the area of the lung tissue for the presence or absence of blood vessels;
creating an opening in lung tissue; and
examining the opening in the lung tissue for the presence or absence of blood vessels.

63. The method of claim 62, where examining the opening in the lung tissue comprises inserting an ultrasound device into the opening in lung tissue to further identify the presence or absence of blood vessels beneath the surface of the lung tissue.

64. The method of claim 62, where examining the area of lung tissue for the presence of blood vessels comprises examining the area at a surface of the lung tissue with the ultrasound device.

65. The method of claim 62, further comprising delivering a fluid to the opening.

66. The method of claim 65, where inserting the ultrasound device into the opening in lung tissue occurs after delivering fluid to the opening.

67. The method of claim 62, where creating an opening in lung tissue comprises creating an opening with the ultrasound device.

68. The method of claim 67, where the ultrasound device further comprises a tissue piercing member.

69. The method of claim 68, where the ultrasound device is configured to blunt the tissue piercing member to prevent the tissue piercing member from damaging tissue.

70. The method of claim 68, where the tissue piercing member comprises a structure selected from the group consisting of a needle, a cannula, a blade, a tube, and a rod.

71. The method of claim 68, where the ultrasound device further comprises an expandable member, and further comprising expanding the opening with the expandable member.

72. The method of claim 71, where the expandable member comprises a balloon.

73. The method of claim 72, where the balloon is a non-distensible balloon.

74. The method of claim 72, where the balloon is a distensible balloon.

75. The method of claim 71, further comprising re-examing the expanded opening for the presence or absence of blood vessels beneath the surface of the lung tissue.

76. The method of claim 71, further comprising delivering an implant to the expanded opening.
